(19) United States
(12) Patent Application Publication
(10) Pub. No.: US 2005/0239747 A1
(43) Pub. Date: Oct. 27, 2005

(54) Compositions and methods of enhanced transdermal delivery of steroid compounds and preparation methods

(75) Inventors: Chih-Chiang Yang, Taipei (TW); Yuan-Chih Le, Taipei Hsien (TW); Chao-Cheng Liu, San Chung City (TW)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Pharmaceutical Industry Technology and Development Center, Wu Ku Hsiang (TW)

(21) Appl. No.: 10/828,700

(22) Filed: Apr. 21, 2004

(51) Int. Cl. A61K 31/724; A61K 31/573; A61K 31/56

(52) U.S. Cl. 514/58; 514/171

(57) ABSTRACT

Enhanced transdermal composition comprising a niosome, which retains within its structure a cyclodextrin inclusion complex of a steroidal active agent and a vesicle of nonionic surfactant are provided herein, which increases the permeability of dermal tissue to transdermally administered steroidal active agents. Methods for producing the enhanced transdermal composition as well as its application are also provided.

### BACKGROUND

1. Field of Invention

The present invention relates in general to a novel pharmaceutical composition and a method for transdermal drug delivery of topical and physiologically active agents and its preparation method thereof. More particularly, the present invention relates to an improved composition for enhanced transdermal penetration of a steroidal active agent and the like using a niosome having a cyclodextrin inclusion complex within its structure; and a method of facilitating the administration of steroidal active agents and the like, and its preparation method thereof.

2. Description of Related Art

The delivery of drugs through the derma, i.e., skin and mucous, provides many advantages over other routes of administration. Primarily, transdermal drug delivery is a comfortable, convenient, and noninvasive way of administrating drugs. Furthermore, a first pass of metabolism, i.e., reduction of metabolism due to an initial bypass of the liver, can be avoided; as are other inherent inconveniences such as gastrointestinal irritation and the like. Transdermal drug delivery also makes possible a high degree of control over blood concentrations of any particular drug. Because of the above listed potential advantages of transdermal drug delivery, the technology in this area is rapidly advancing and researchers have long been searching an effective means of introducing drugs into the systemic circulation by applying them to the unbroken skin or mucous membrane. Many problems have been encountered when attempting to deliver drug through transdermal route. For example, some medicaments do not dissolve easily and therefore require higher doses.

It is known that various substances can enhance the ability of drugs and agents to diffuse through the skin and other tissues. The more popular approach has been the employment of surface active agents. However, many surface active agents enhance the permeability by actually damaging the barrier tissue. Only slight to moderate enhancement of penetration is effected with the prior art surface active agents.

Another approach is the use of certain organic solvents. U.S. Pat. Nos. 4,006,218, 3,551,554 and 3,472,931 respectively describe the use of dimethylsulfoxide (DMSO), dimethyl formamide (DMF) and N,N-dimethylacetamide for enhancing the penetration of active substances through stratum corneum. A disadvantage of using these solvents is that they are systemically distributed in a short period of time and cause undesirable side effects.

Transdermal delivery of steroidal active agents is provided in the technical documents listed below. For example, U.S. Pat. No. 4,435,180 describes a system for the transdermal administration of progesterone. Concurrent administration of progesterone and estradiol esters is described in U.S. Pat. No. 4,788,062. U.S. Pat. No. 6,555,131 provides a therapeutical system for transdermal delivery of sex steroid hormones; and U.S. Pat. No. 4,973,468 of Chiang discloses the use of a skin permeation enhancer composition comprising an ether component and an ester component in conjunction with the transdermal administration of steroid drugs such as progestogens and estrogens.

There exists a need of an improved composition for transdermal delivery of steroidal active agents and a method of enhancing transdermal and transmucosal penetration of steroidal active substances without adverse side effects, locally or systemically, on the skin or body membranes.

Inventors of this application have identified a novel formulation and method of enhanced transdermal delivery of steroidal active agents by use of a cyclodextrin inclusion complex in the transdermal formulation. Natural cyclodextrins and their derivatives have been used in the pharmaceutical industry for solubilising sparingly water soluble drugs to enhance their bioavailability. Specifically, cyclodextrins are cyclic oligosaccharides consisting of glucose units fused together as a ring. The primary and secondary hydroxyl groups of the glucose units are present on the exterior of the cyclodextrin molecule thus making the exterior part hydrophilic, whereas the interior of the cyclodextrin molecule is hydrophorbic. This amphiphatic property of cyclodextrin makes it an ideal molecule for solubilising water insoluble compounds by forming inclusion complexes. α, β, and γ-cyclodextrins are the three naturally occurring forms consisting of 6, 7 and 8 glucose units respectively. Among these, β-cyclodextrin is the most abundant and commonly used one, it is also currently the only cyclodextrin which is Generally Recognized As Safe (GRAS) for foods. Several synthetic derivatives of cyclodextrins such as methyl, propyl, isopropyl, hydroxy methyl, hydroxy ethyl, hydroxy propyl and sulfoalkyl, have also been used to enhance their solubility in water. Technical documents concerning cyclodextrin inclusion complex and its applications are listed below and their contents are incorporated herein by reference. For example, European Patent No. 1191851 B1; WO 01/85218; WO 03/043662; WO 98/42382; WO 03/059393; WO 98/02186; WO 00/54596; WO 01/10913; WO 00/47811 and WO 00/53637.

To date, there has never been an attempt of enhancing the transdermal permeation of steroidal active agents by use of niosome having a cyclodextrin inclusion complex within its structure. Surprisingly, the novel enhanced transdermal formulation according to this application greatly enhances the penetration of steroid compounds across the biomembranes.

### SUMMARY

Accordingly, It is the objectives of this invention to provide an improved composition for transdermal application and its preparation method thereof; and a method of enhancing transdermal and transmucosal penetration of steroidal active substances by use of a niosome comprising a cyclodextrin inclusion complex.

It is therefore an objective of the present application to provide a pharmaceutical composition comprising a niosome, which retains within its structure:
(1) a cyclodextrin inclusion complex formed by a cyclodextrin compound and a steroidal active agent; and
(2) a vesicle formed by a nonionic surfactant;
wherein said niosome can facilitate the transdermal delivery of said steroidal active agent.

It is another objective of the present invention to provide a method for producing a composition comprising a niosome, wherein the niosome retaining within its structure:
(1) a cyclodextrin inclusion complex formed by a cyclodextrin compound and a steroidal active agent; and
(2) a vesicle formed by a nonionic surfactant;
wherein said niosome can facilitate the transdermal delivery of said steroidal active agent,
the method comprising the steps of:
(a) forming a cyclodextrin inclusion complex of a steroidal active agent;
(b) forming a vesicle solution of a nonionic surfactant;
(c) mixing the vesicle solution of step (b) with the cyclodextrin inclusion complex of step (a) in a molar ratio of about 1.0 to 25.0; and
(d) drying the resulted mixture of step (c).

It is still another objective of the present invention to provide a method for facilitating transdermal delivery of a steroidal active agent, comprising administering to a human or an animal a composition comprising a niosome, which retains within its structure a cyclodextrin inclusion complex of a steroidal agent and a vesicle formed by a nonionic surfactant.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the preferred embodiment, with reference made to the accompanying drawings as follows:

FIG. 1 is graphic representation of the IR spectra of free estriol, β-cyclodextrin and the cyclodextrin inclusion complexes of Examples 1 and 2, respectively;

FIG. 2 is a graphic representation of the Differential Scanning Calorimetry (DSC) analysis of free estriol, β-cyclodextrin and the cyclodextrin inclusion complexes of Examples 1 and 2;

FIG. 3 illustrates the permeation kinetics of free estriol and the niosomes of Examples 5 and 6; and

FIG. 4 illustrates the permeation kinetics of free estriol and the niosomes of Example 6 with the molar ratio of Span 60: the cyclodextrin inclusion complex of estriol=1:1, 5:1, and 13.5:1, respectively.

### DESCRIPTION OF THE PREFERRED EMBODIMENTS

By “transdermal” delivery, applicants intended to include both transdermal (or “percutaneous”) and transmucosal administration, i.e., delivery by passage of a drug through the skin or mucosal tissue and into the bloodstream.

“Enhanced delivery”, “enhanced permeability”, “enhanced permeation” or “enhanced penetration” as used herein relates to an increase in the permeability of skin or mucous membrane to a pharmacologically active agent, i.e., so as to increase the rate at which the drug permeates through the skin or mucous membrane and enters the bloodstream. The enhanced permeation effected through the use of the composition of the present invention can be observed by measuring the rate of diffusion of a drug through animal or human skin using a diffusion cell apparatus as described in the Examples herein.

By the term “a steroidal active agent” as used herein is meant any steroid compound, its analogs or derivatives suitable for transdermal or transmucosal administration which induced a desired systemic effect. Examples of steroidal active agents useful herein include: progestogens such as norethindrone, norethindrone acetate, desogestrel, 3-keto desogestrel, gestadene and levonogestrel; estrogens such as estradiol, estriol and its esters; corticosteroids such as cortisone, hydrocortisone, and fluocinolone acetonide; and androgens such as testosterone.

In a preferred embodiment, the composition of this invention comprises a niosome, the niosome comprises within its structure a vesicle formed by a nonionic surfactant and a cyclodextrin inclusion complex in a molar ratio of about 0.5 to 30; and more preferably, about 1.0 to 25.

Examples of cyclodextrins useful herein may be natural cyclodextrins or their synthetic derivatives or combination thereof. The natural cyclodextrins are α, β, and γ-cyclodextrins and the synthetic derivatives are methyl, propyl, isopropyl, hydroxy methyl, hydroxy ethyl, hydroxy propyl and sulfoalkyl derivatives. The method of the present invention has been found to work best with β-cyclodextrin because it increases the efficiency in forming complexes. Suitably, the cyclodextrin that is combined with other components is in liquid from, e.g., an aqueous slurry of cyclodextrin.

The present invention also provides a method of producing a composition as described above comprising the incorporation into a niosome of a cyclodextrin inclusion complex and a vesicle formed by a nonionic surfactant.

According to one embodiment of this present invention provides a method comprising the steps of:
a) forming a cyclodextrin inclusion complex of a steroidal active agent;
b) forming a vesicle solution of a nonionic surfactant;
c) mixing the vesicle solution of step (b) with the cyclodextrin inclusion complex of step (a) in a molar ratio of about 1.0 to 25.0; and
d) drying the resulted mixture of step (c).

In step a), the cyclodextrin inclusion complex may be formed by a physical mixing process or a freeze-drying process as described in Examples herein. The molar ratio of cyclodextrins and steroidal active agents is preferably about 0.5 to 15; more preferably, about 1.0 to 10.0.

In step b), the vesicle solution of a nonionic surfactant is formed by dissolving a nonionic surfactant in alcohol at a temperature between 40° C. to 60° C., followed by injecting the alcohol containing mixture into stirring water of same temperature with a syringe.

In step c), the vesicle solution of step (b) is mixed with the cyclodextrin inclusion complex of step (a) in a molar ratio of about 1.0 to 25.0 to form a niosome slurry; and the niosomes thus formed may be freeze dried and/or spray dried (in step (d)) and the resulting pellets or powder stored until further required when they can be rehydrated.

Examples of the nonionic surfactants useful herein include Span series such as Span 20, Span 40, Span 60, and Span 80; Tween series (polyoxyalkylene sorbitan monostearate type surfactant) such as Tween 20, Tween 40, Tween 60 and Tween 80; alcohols such as α-diols, C₁-C₂₀ alkylphenols; polyalkylene oxide derivatives such as polyethylene oxide and copolymers of ethylene and propylene oxide, condensates of ethylene and propylene oxide with fatty alcohols, polyethoxylated fatty amides preferably having from 2 to 30 mol of ethylene oxide, polyglycerolated fatty amides comprising on average 1 to 5 glycerol groups and in particular 1.5 to 4, polyethoxylated fatty amines preferably having 2 to 30 mol of ethylene oxide, ethoxylated fatty acid esters of sorbitan having from 2 to 30 mol of ethylene oxide; polyethoxylated, polypropoxylated or polyglycerolated fatty acids having a fatty chain of C₈-C₁₈ carbon atoms, and the number of ethylene oxide or propylene oxide groups ranges from 2 to 50, and the number of glycerol groups ranges in particular from 2 to 30; fatty acid esters of sucrose; fatty acid esters of polyethylene glycol; (C₆-C₂₄)alkyl polyglycosides; derivatives of N—(C₆-C₂₄)alkyl glucamine; amine oxides such as (C₁₀-C₁₄)alkylamine oxides or N—(C₁₀-C₁₄)acylaminopropylmorpholine oxides; and related compounds and combinations thereof. The particular suitable nonionic surfactant was from Span series offered by Showa Chemicals Inc. (Kawasaki, Japan) and more specifically the Span 60 surfactant, which is a sorbitol monostearate type surfactant. The use of Span 60 surfactant results in niosomes with enhanced permeability across the biomembranes.

The compositions in accordance with this invention can be formulated in a variety of dosage forms for topical application. A wide variety of dermatologically acceptable inert carriers well known to the art may be employed. The topical compositions may include liquids, creams, lotions, ointments, gels, sprays, aerosols, skin patches, and the like. Typical inert carriers could be, for example, water, ethyl alcohol, polyvinyl pyrrolidone, propylene glycol, mineral oil, stearyl alcohol and gel-producing substances. All of the above dosages forms and carriers are well known to the cosmetic and pharmaceutical art. The choice of the dosage form is not critical to the efficacy of the composition described herein.

The compositions in accordance with this invention can also be formulated in a variety of dosage forms for mucosal application, such as buccal and/or sublingual drug dosage units for drug delivery through oral mucosal membranes. A wide variety of bioerodible polymeric carriers may be used that are pharmaceutically acceptable, provide both a suitable degree of adhesion and the desired drug release profile, and are compatible with the active agents to be administered and any other components that may be present in the buccal and/or sublingual drug dosage units. Generally, the polymeric carrier comprises hydrophilic polymers that adhere to the wet surface of the oral mucosa. Examples of polymeric carriers include, but not limited to, acrylic acid polymers and copolymers; hydrolyzed polyvinylalcohol; polyethylene oxides; polyacrylates; vinyl polymers and copolymers; polyvinylpyrrolidone; dextran; guar gum; pectins; starches; and cellulosic polymers.

The compositions in accordance with this invention can also be formulated in an inhaler aerosol formulation for drug delivery through nasal mucosal membranes. Suitable propellants and/or co-solvents for solubilizing the active agents in medicinal aerosol formulations are well known in this art. Typical propellants are hydrofluoroalkanes such as 1,1,1,2-tetrafluoroethane (HFA-134a), 1,1,1,2,3,3,3-heptafluoropropane (HFA-227ea). pentafluoroethane (HFA-125), 1,1-difluoroethane (HFA-152a), difluoromethane (HFA-32) and the like. Typical co-solvents include, but not limited to, alcohols, polyols, alkoxy derivatives, fatty acid alkyl esters, polyalkylene glycols, dimethylsulphoxide and the like.

Accordingly, the present invention further provides a method of facilitating transdermal delivery of a steroidal active agent, comprising administering to a human or an animal the composition prepared as described herein.

Reference will now be made in detail to the present preferred embodiments of the invention, examples of which are illustrated in the accompanying drawings. Further embodiments will occur to those skilled in the art in the light of these.

Preparation of Cyclodextrin Inclusion Complex

### EXAMPLE 1

### Preparation of Cyclodextrin Inclusion Complex of Estriol by Physical Mixing Process

In a grinder, weighted and mixed β-cyclodextrin and estriol in a ratio of 9.84 to 1 by weight (or 2.5 to 1 by molarity), and grounded the mixture until it became homogenous.

### EXAMPLE 2

### Preparation of Cyclodextrin Inclusion Complex of Estriol by Freeze-Drying Process

To a water solution of β-cyclodextrin of 40-50° C., an estriol ethanol solution was added, wherein the molar ratio of β-cyclodextrin and estriol in the mixed solution was between 2.5 to 1. After thorough mixing of the two solutions, the solvent was evaporated under reduced pressure and the product was freeze-dried.

Characterization of the Cyclodextrin Inclusion Complexes of Examples 1 and 2

The cyclodextrin inclusion complexes of Examples 1 and 2 were characterized by IR spectroscopy and differential scanning calorimetry (DSC) analysis.

### EXAMPLE 3

### IR Spectroscopy

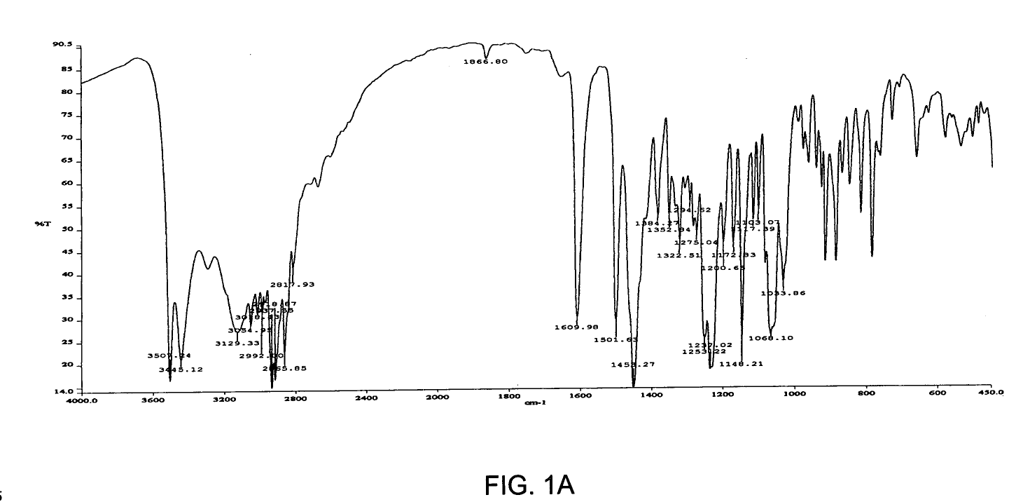

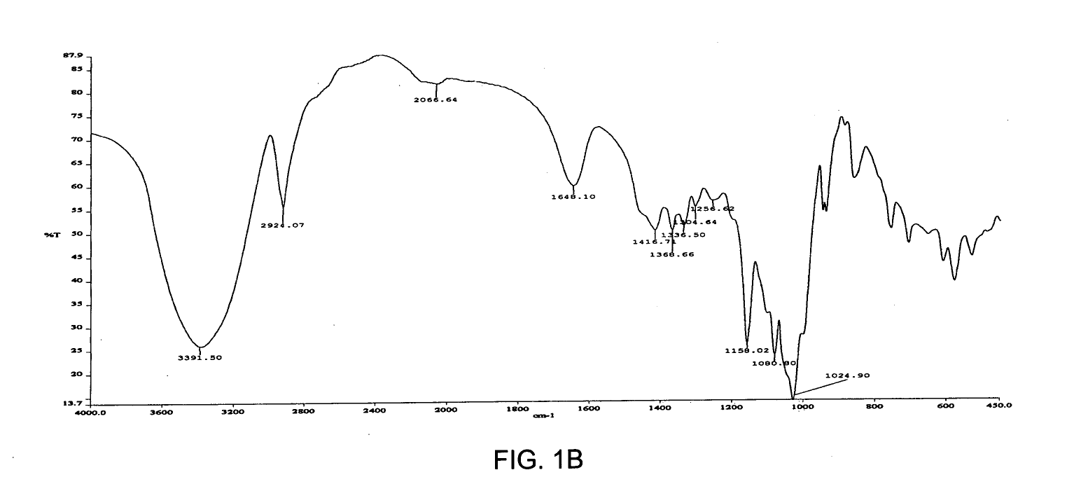

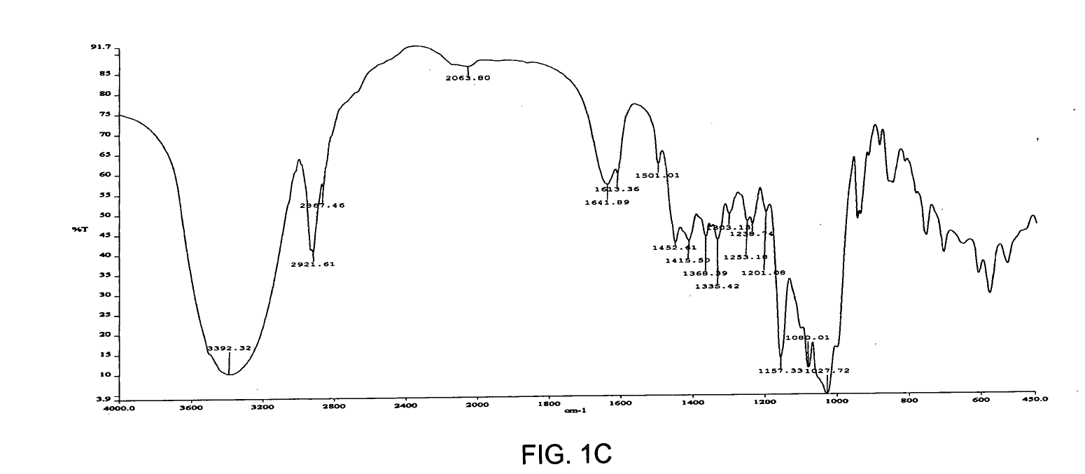

The cyclodextrin inclusion complexes of Example 1 or 2 were grounded in a grinder with potassium bromide (KBr) and pressed into a pellet for IR analysis. Results were shown in FIG. 1. Free estriol has maximum absorptions in IR range of 3507, 2855 and 1455 cm⁻¹ as illustrated in FIG. 1A, and with estriol being incorporated into β-cyclodextrin, there is a resultant change in absorbance at 1455, 1384 and 1103 cm⁻¹ as shown in FIGS. 1C (Example 1) and 1D (Example 2).

### EXAMPLE 4

### Differential Scanning Calorimetry (DSC) Analysis

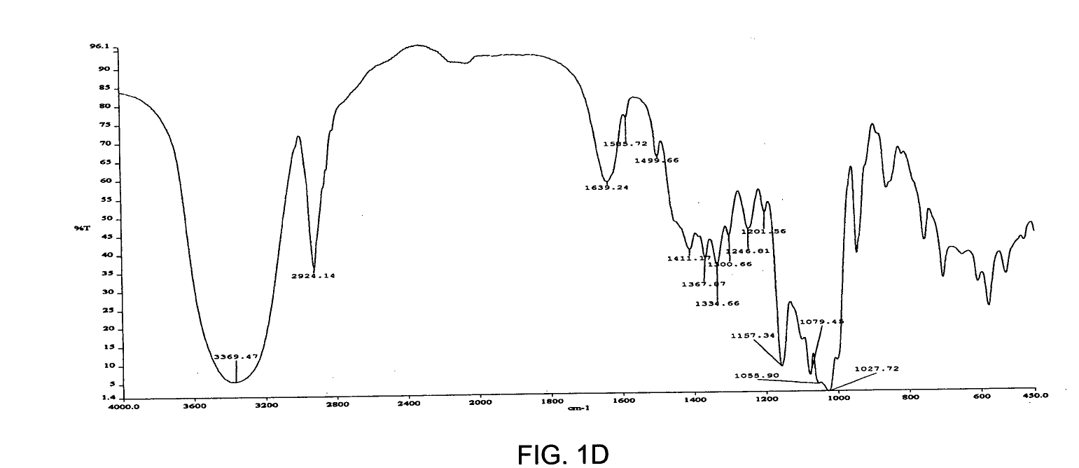

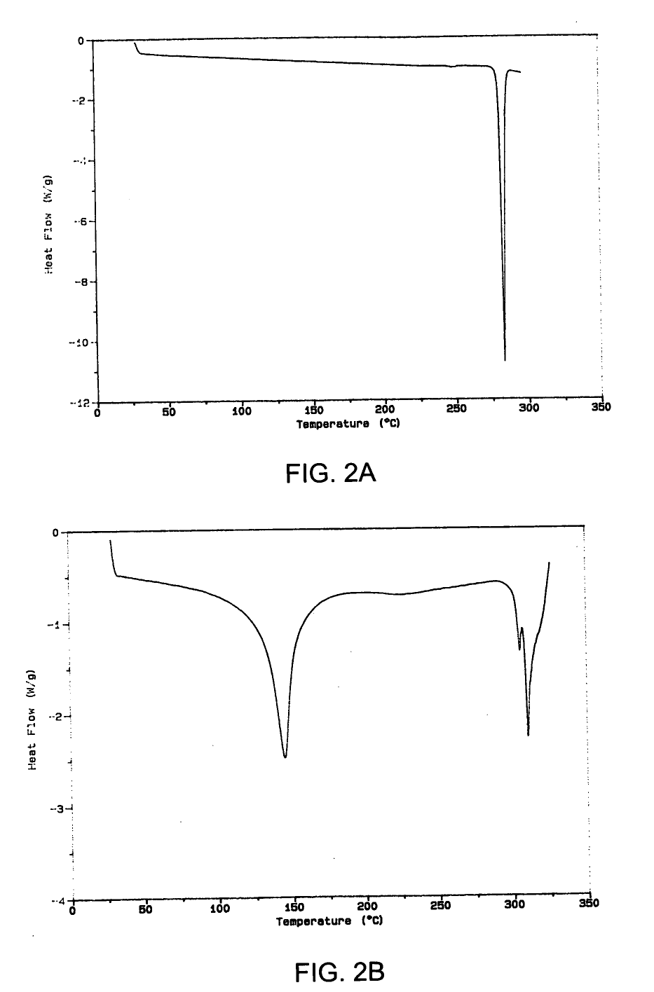

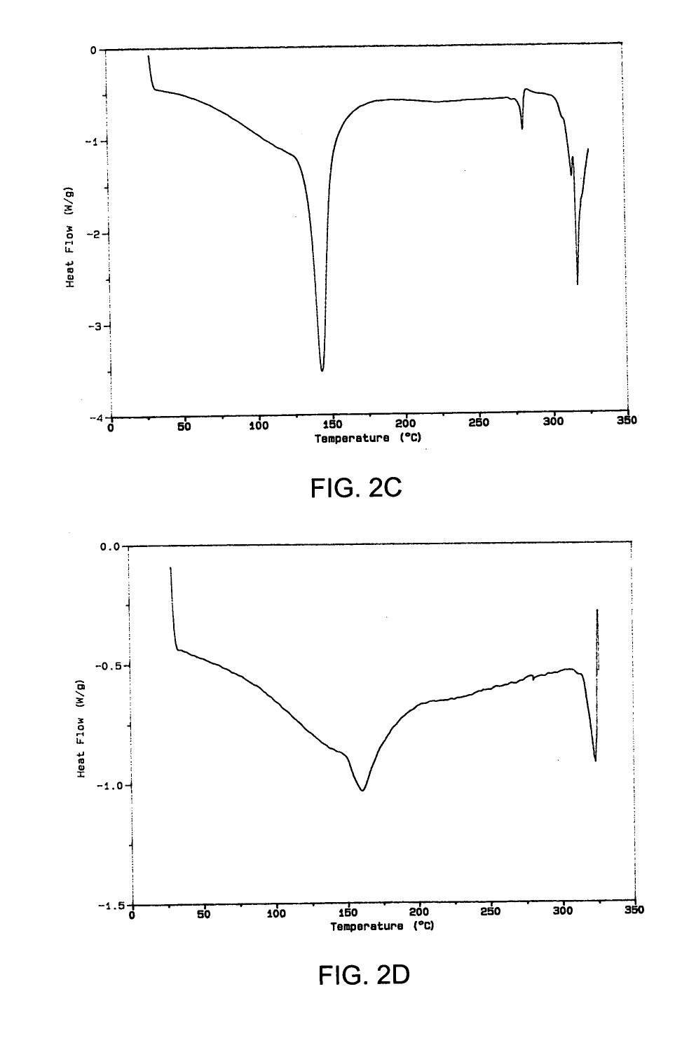

The cyclodextrin inclusion complexes of Example 1 or 2 were grounded in a grinder and pressed into a pellet for DSC analysis. DSC apparatus was first calibrated with indium and purged with helium at a flow rate of 40 ml/min. The test samples were first heated from a temperature of 50° C. at a rate of 10° C./min until it reached 105° C., then the temperature was maintained at 105° C. for 5 minutes to ensure complete removal of moisture. Let the test samples stand still and when their temperature returned to room temperature, they were reheated again from 30° C. at a rate of 10° C./min until the temperature reached 320° C. Results were illustrated in FIG. 2. Free estriol characterizes in having an endothermic transition melting point at around 280° C. as shown in FIG. 2A. β-cyclodextrin, on the other hand, characterizes in having two endothermic transition melting points at around 145° C. and 310° C., respectively (FIG. 2B). The inclusion complexes prepared by physical mixing process (Example 1) possessed similar endothermic transition melting points as that of β-cyclodextrin (FIG. 2C). However, the inclusion complexes prepared by freeze-drying process (Example 2) showed an increase of heat flow over a rise in temperature and the lower endothermic transition melting point was gradually shifted from 145° C. to around 160° C. with the higher endothermic transition melting point remained relatively unchanged (FIG. 2D).

Preparation of Niosomes

### EXAMPLE 5

### Preparation of Niosomes Comprising Span 60 and Estriol (Span(E))

Dissolve proper amounts of Span 60 or estriol in ethanol at a temperature of about 50° C., respectively. The molar ratio of Span 60 and estriol used is 13.38 to 1. At the same time, prepared a water solution with its volume at least 10 times greater than that of the ethanol solution used above, and the water solution was heated to about 50° C. To this hot water solution, rapidly added in the ethanol solutions of Sapn 60 and/or estriol described above respectively, with a syringe. Solvent of the mixed solution was removed by reduced pressure concentration and followed by freeze-drying and/or spray-drying to generate niosomes that contain both Span 60 and estriol.

Rehydration When in use, the freeze-dried niosome powders were rehydrated by dissolving in solution A (composition: 120 mg estriol, 30 g 0.1 N NaOH, 6.0 g glycerin, 1.2 g benzyl alcohol, filled up with water to 90 g, adjusted acidity by 5% lactic acid until pH value reached 4.0). The final estriol concentration was around 1,000 ppm.

### EXAMPLE 6

### Preparation of Niosomes Comprising Span 60 and the Cyclodextrin Inclusion Complex of Estriol (Span(CD/E))

The method of Example 5 was repeated except β-cyclodextrin inclusion complex of estriol instead of free estriol was used (the molar ration of Span 60, cyclodextrin and estriol is 13.38:2.5:1.0, the weight ratio is 20:9.84:1). The rehydration procedure is the same as described above.

Characterization of the Niosomes of Examples 5 and 6

The niosomes of Examples 5 and 6 were characterized by entrapment analysis.

### EXAMPLE 7

### Entrapment Analysis

The niosomes of Example 5 or 6 were rehydrated in solution A at 25° C., 57° C., and 70° C. respectively, according to the procedure described above. 1 ml of the rehydrated niosomes was placed it in a microcentrifuge tube and centrifuged in a speed of 13,000 rpm at room temperature for 10 minutes. After centrifugation, collected the upper clear solution (contained free estriol) and precipitated niosomes, respectively. The precipitated niosomes were rehydrated with 1 ml of Solution A as described above. The amount of estriol entrapped in both the upper clear solution and in the niosome precipitate were calculate and expressed in the percentage of estriol entrapped in the niosomes. Result was summarized in table 1. The amount of estriol entrapped within the niosomes containing both Span 60 and the cyclodextrin inclusion complexes of estriol (Example 6) is around 54%, and decreases with an increase in temperature. On the contrary, the amount of estriol entrapped within the niosome is highest in niosomes containing just Span 60 and estriol (Example 5), over 90%, and the entrapped percentage varies slightly with temperature.

TABLE 1
The effect of temperature on the efficacy of estriol entrapped within the
niosomes of Example 5 and 6

| | Percentage of | | |
|---|---|---|---|
| | entrapment (%) | | |
| Niosomes | 25° C. | 57° C. | 75° C. |
| Example 5 | 93.42 | 95.21 | 87.75 |
| Example 6 | 54.49 | 9.29 | 10.21 |

Enhanced Permeation Analysis on the Niosomes of Examples 5 and 6 Example 8 Permeation Analysis Using Franz Diffusion Cells

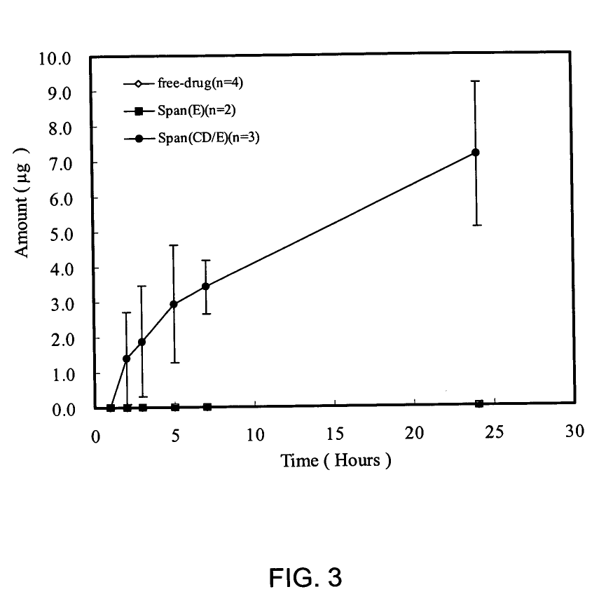

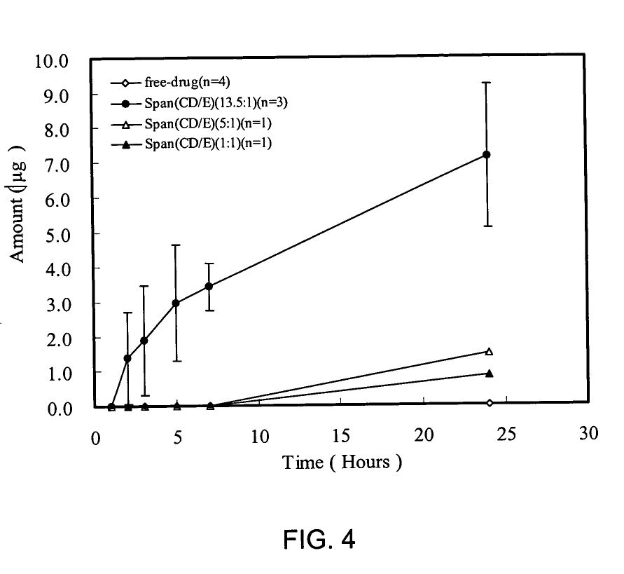

In vitro Franz Diffusion cells were used to compare the penetration of niosomes of Examples 5 and 6. Briefly, a piece of human skin tissue (a generous gift from Kaohsiung Medical University (Kaohsiung, Taiwan) was mounted between the two half-cells and fastened with a clamp. Aliquots of free estriol and/or niosomes (about 500 ppm) were applied to the Donor compartment to start the experiment. The receiver compartment was filled with PBS and the temperature was held at 37° C. Samples were taken at preset time intervals, specifically at 1, 2, 3, 4, 6, 8, 10, and 24 hours respectively, and assayed by HPLC. Amounts of estriol accumulated in the receiver compartment were calculated and results were summarized in FIGS. 3 and 4.

FIG. 3 illustrated the permeation kinetics of free estriol and niosome of Examples 5 and 6. An increase in skin permeation rate through the entire timeframe was observed when the niosomes containing cyclodextrin inclusion complexes of estriol (Example 6) were used. More importantly, an enhanced skin permeation was achieved with the inclusion of cyclodextrin complex in the niosomes, the permeation rate increased by about 2-7 folds compared with either the free estriol or the niosomes containing estriol (Example 5). Furthermore, the inventors of this application also identified that the permeation rate of the niosomes is affected by the amount of Span 60 being incorporated in the niosomes. As illustrated in FIG. 4, as the molar ratio of Span 60 to cyclodextrin inclusion complex of estriol increases from 1:1 to 13.5:1, the permeation rate of the niosomes increases dramatically over time and reaches a maximum of 7 folds by 24 hours.

Effects of the Invention

As described hereinabove, niosome having a cyclodextrin inclusion complex within its structure may enhance the transmembrane delivery of a steroid compound, which makes said niosome an useful agent in pharmaceutical industry as well as cosmetic industry. The cyclodextrin containing niosome according to the present invention can be used as a tool for facilitating the delivery of active agents, particularly, steroidal active agents, across biomembranes.

Although the present invention has been described in considerable detail with reference to preferred embodiments thereof, however, other embodiments are possible for those skilled in the art and various modifications and variations can be made to the niosome of the present invention without departing from the scope or spirit of the invention. In view of the foregoing, it is intended that the present invention cover modifications and variations of this invention provided they fall within the scope of the following claims and their equivalents.

1. A composition comprising a niosome, wherein the niosome retaining within its structure:
(1) a cyclodextrin inclusion complex formed by a cyclodextrin compound and a steroidal active agent; and
(2) a vesicle formed by a nonionic surfactant;
wherein said niosome can facilitate the transdermal delivery of said steroidal active agent.

2. The composition of claim 1, wherein said steroidal active agent is selected from the group consisting of progestogens, corticosteroids, estrogens, and androgens.

3. The composition of claim 1, wherein said cyclodextrin compound is selected form the group consisting of α-cyclodextrin, β-cyclodextrin, γ-cyclodextrin, methyl-cyclodextrin, propyl-cyclodextrin, isopropyl-cyclodextrin, hydroxy-cyclodextrin, hydroxyethyl-cyclodextrin, hydroxypropyl-cyclodextrin, and sulfoalkyl-cyclodextrin.

4. The composition of claim 3, wherein said cyclodextrin compound is β-cyclodextrin.

5. The composition of claim 1, wherein said cyclodextrin inclusion complex is comprised of a cyclodextrin compound and a steroidal active agent in a molar ratio of about 1.0 to 10.0.

6. The composition of claim 1, wherein said nonionic surfactant is selected from the group consisting of alcohols, polyalkylene oxide derivatives, alkyl polyglycosides, derivatives of N-alkyl glucamine; fatty acid esters of sucrose; fatty acid esters of polyethylene glycol; (C₆-C₂₄)alkyl polyglycosides; derivatives of N—(C₆-C₂₄)alkyl glucamine; amine oxides; sorbitol monostearate type surfactant and polyoxyalkylene sorbitan monostearate type surfactant.

7. The composition of claim 6, wherein said nonionic surfactant is a sorbitol monostearate type surfactant.

8. The composition of claim 1, wherein said niosome is comprised of a nonionic surfactant vesicle and a cyclodextrin inclusion complex in a ratio of 1.0 to 25.0.

9. A method of producing a composition comprising a niosome, wherein the niosome retaining within its structure:
(1) a cyclodextrin inclusion complex formed by a cyclodextrin compound and a steroidal active agent; and
(2) a vesicle formed by a nonionic surfactant;
wherein said niosome can facilitate the transdermal and/or transmucosal delivery of said steroidal active agent,
the method comprising the steps of:
(a) forming a cyclodextrin inclusion complex of a steroidal active agent;
(b) forming a vesicle solution of a nonionic surfactant;
(c) mixing the vesicle solution of step (b) with the cyclodextrin inclusion complex of step (a) in a molar ratio of about 1.0 to 25.0; and
(d) drying the resulted mixture of step (c).

10. The method of claim 9, wherein said steroidal active agent is selected from the group consisting of progestogens, corticosteroids, estrogens, and androgens.

11. The method of claim 9, wherein said cyclodextrin compound is selected form the group consisting of α-cyclodextrin, β-cyclodextrin, γ-cyclodextrin, methyl-cyclodextrin, propyl-cyclodextrin, isopropyl-cyclodextrin, hydroxy-cyclodextrin, hydroxyethyl-cyclodextrin, hydroxypropyl-cyclodextrin, and sulfoalkyl-cyclodextrin.

12. The method of claim 11, wherein said cyclodextrin compound is β-cyclodextrin.

13. The composition of claim 9, wherein said cyclodextrin inclusion complex is comprised of a cyclodextrin compound and a steroidal active agent in a molar ratio of about 1.0 to 10.0.

14. The method of claim 9, wherein said nonionic surfactant is selected from the group consisting of alcohols, polyalkylene oxide derivatives, alkyl polyglycosides, derivatives of N-alkyl glucamine; fatty acid esters of sucrose; fatty acid esters of polyethylene glycol; (C₆-C₂₄)alkyl polyglycosides; derivatives of N—(C₆-C₂₄)alkyl glucamine; amine oxides; sorbitol monostearate type surfactant and polyoxyalkylene sorbitan monostearate type surfactant.

15. The method of claim 14, wherein said nonionic surfactant is a sorbitol monostearate type surfactant.

16. The method of claim 9, wherein said mixing of step (a) is a physical mixing process or a freeze-drying process.

17. The method of claim 16, wherein the physical mixing process characterized in grinding a mixture of a cyclodextrin compound and a steroidal active agent in a grinder until the mixture is homogenous.

18. The method of claim 16, wherein the freeze-drying process characterized in having the steps of:
(a) mixing an aqueous solution of a cyclodextrin compound and an alcoholic solution of a steroidal active agent;
(b) evaporating the solvent of said solution mixture of step (a); and
(c) freeze-drying the resulted mixture of step (b).

19. A method for facilitating transdermal delivery of a steroidal active agent, comprising administering to a human or an animal the composition of claim 1.
